(19) United States
(12) Plant Patent Application Publication
(10) Pub. No.: US 2002/0100100 P1
(43) Pub. Date: Jul. 25, 2002

(54) Guinea impatiens plant named ' Kimbu '

(76) Inventor: Norbert Bull, Goennebek (DE)

Correspondence Address: Catherine Anne Whealy, 1513 Melody Lane, Roanoke, TX, 76262, US

(21) Appl. No.: 09/765,323

(22) Filed: Jan. 22, 2001

(51) Int. Cl. A01H 5/00

(52) U.S. Cl. PLT/318

(57) ABSTRACT

A new and distinct cultivar of New Guinea Impatiens plant named ‘Kimbu’, characterized by its soft and dark orange bi-colored flowers; moderately large round flowers that are positioned well above and beyond the foliage; freely flowering habit; early to medium flowering response; compact, low-growing and uniformly mounded plant growth habit; dark green-colored leaves; and resistance to Powdery Mildew.

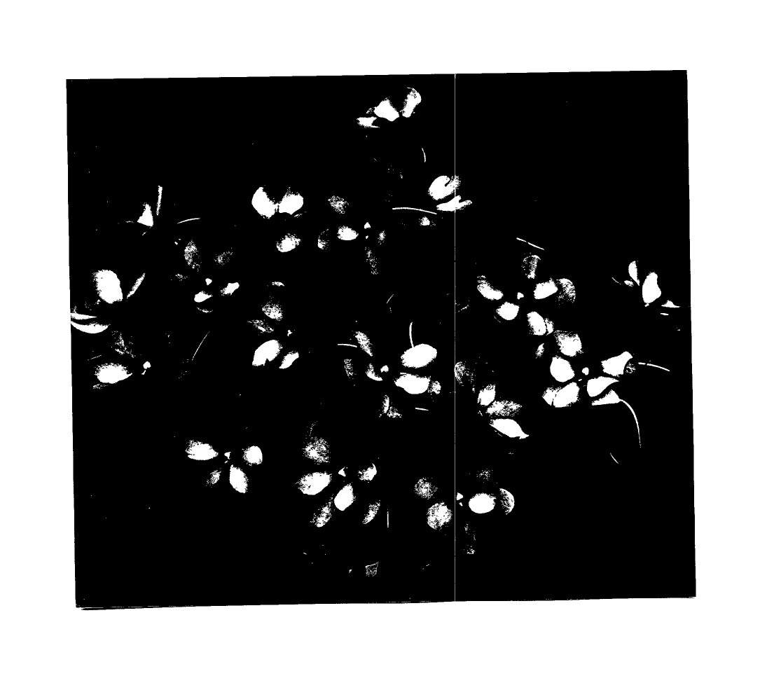

### BACKGROUND OF THE INVENTION

The present Invention relates to a new and distinct cultivar of New Guinea Impatiens plant, botanically known as Impatiens hawkeri, and hereinafter referred to by the name ‘Kimbu’.

The new Impatiens is a product of a planned breeding program conducted by the Inventor in Goennebek, Germany. The objective of the breeding program is to develop new Impatiens cultivars with large rounded bi-colored flowers and dark-colored foliage.

The new Impatiens originated from a cross made by the Inventor in 1997 of an Impatiens hawkeri seedling selection designated as code number 4, not patented, as the male, or pollen parent, with an Impatiens hawkeri seedling selection designated as code number 18, not patented, as the female, or seed parent. Both selections are proprietary and used only in the Inventor's breeding program. The cultivar Kimbu was discovered and selected by the Inventor as a flowering plant within the progeny of the stated cross in a controlled environment in Goennebek, Germany, in the fall of 1997. Plants of the new Impatiens differ from plants of the parent selections primarily in flower and foliage color.

Asexual reproduction of the new cultivar by terminal cuttings taken in Goennebek, Germany, by, or under the supervision of the Inventor, since the fall of 1997, has shown that the unique features of this new Impatiens are stable and reproduced true to type in successive generations.

### SUMMARY OF THE INVENTION

The following traits have been repeatedly observed and are determined to be the unique characteristics of ‘Kimbu’. These characteristics in combination distinguish ‘Kimbu’ as a new and distinct Impatiens cultivar:

1. Soft and dark orange bi-colored flowers.

2. Moderately large round flowers that are positioned well above and beyond the foliage.

3. Freely flowering habit.

4. Early to medium flowering response.

5. Compact, low-growing and uniformly mounded plant growth habit.

6. Dark green-colored leaves.

7. Resistant to Powdery Mildew.

Plants of the new Impatiens can be compared to plants of the cultivar Sumba, not patented. However, in side-by-side comparisons conducted by the Inventor in Goennebek, Germany, plants of the new Impatiens differ from plants of the cultivar Sumba in the following characteristics:

1. Plants of the new Impatiens have dark red-colored pedicels whereas plants of the cultivar Sumba have light green-colored pedicels with slight red infusion.

2. Petals of plants of the new Impatiens are more rounded in shape and apices are not as deeply incised as petals of plants of the cultivar Sumba.

3. The soft orange petal color of plants of the new Impatiens is darker than the soft orange petal color of plants of the cultivar Sumba.

### BRIEF DESCRIPTION OF THE PHOTOGRAPH

The accompanying colored photograph illustrates the overall appearance of the new cultivar, showing the colors as true as it is reasonably possible to obtain in colored reproductions of this type. Colors in the photograph may differ slightly from the color values cited in the detailed botanical description which accurately describe the colors of the new Impatiens. The photograph comprises a top perspective view of a typical flowering plant of ‘Kimbu’ grown in a container.

### DETAILED BOTANICAL DESCRIPTION

The cultivar Kimbu has not been observed under all possible environmental conditions. The phenotype may vary somewhat with variations in environment such as temperature, light intensity, daylength, water status and/or fertility level, without, however, any variance in genotype.

The following observations and measurements describe plants grown in Hillscheid, Germany, under commercial practice in a glass-covered greenhouse. Two groups of plants were used for the following description: one group was grown during the spring of 1999 and the other group was grown during the spring of 2000. Both groups of plants were planted in early March in 12-cm containers and grown with 20° C. minimum day/night temperatures. The following observations and measurements were taken when the plants started flowering or about 10 weeks after planting rooted cuttings. In the following description, color references are made to The Royal Horticultural Society Colour Chart except where general terms of ordinary dictionary significance are used.

Botanical Classification: Impatiens hawkeri cultivar Kimbu.

Commercial Classification: New Guinea Impatiens cultivar Kimbu.

Parentage:

Male parent.—Inventor's proprietary Impatiens hawkeri seedling selection designated as code number 4, not patented.

Female parent.—Inventor's proprietary Impatiens hawkeri seedling selection designated as code number 18, not patented.

Propagation:

Type cutting.—Terminal tip cuttings.

Time to produce rooted cuttings.—About 18 days at 22° C.

Root description.—Numerous, fibrous, and freely branching.

Plant Description:

General appearance.—Compact, low-growing and uniformly mounded plant growth habit; freely branching.

Crop time.—From a rooted cutting, about 10 weeks are required to produce finished flowering plants in 12-cm containers.

Plant height.—About 14 cm.

Plant diameter or spread.—About 25 cm.

Lateral branches.—Internode length: About 2.5 to 3.5 cm. Color: Dark purple, 185A, with some green undertones.

Foliage description.—Arrangement: Primarily in whorls. Length: About 10 cm. Width: About 3.5 cm. Shape: Elliptic. Apex: Slightly acuminate. Base: Acute. Margin: Serrulate with ciliation. Texture: Smooth. Color: Upper surface: Dark green, close to 139A to 147A, slightly glossy; venation, 185B. Lower surface: Dark purple, close to 183A; venation, 185A. Petiole: Length: About 1 to 1.5 cm. Color: Upper surface: 185B. Lower surface: 185A.

Flower Description:

Flower type and flowering habit.—Single rounded soft and dark orange bi-colored flowers. Freely flowering, usually about 8 flowers and flower buds per lateral branch. Flowers positioned well above and beyond the foliage and typically face upward or outward. Petals self-cleaning; gynoecium persistent. Flowers not fragrant.

Flower longevity.—Flowers last about 3 weeks on the plant.

Flowering season.—Year-round under greenhouse conditions. In the garden, flowering from spring until fall.

Flower length.—About 5.9 cm.

Flower width.—About 5.9 cm.

Petals.—Quantity: Single, five per flower, imbricate. Length: Banner petal: About 2.5 cm. Lateral and base petals: About 2.8 cm. Width: Banner petal: About 4.6 cm. Lateral and base petals: About 3.6 cm. Shape: Roughly cordate. Apex: Emarginate, slightly lobed. Base: Attenuate. Margin: Entire. Aspect: Mostly flat. Texture: Smooth; velvety. Color: When opening and filly opened, upper surface: Banner petal: Central apical splotch, orange, 33A to 40A; surrounded by soft orange, 33B to 33C, to margins; base, 46B. Lateral and base petals: Central stripe, orange, 33A to 40A; surrounded by soft orange, 33B to 33C, to margins; base, 46B. When opening and fully opened, lower surface: Orange, 40B to 40C.

Spur.—Quantity per flower: About 1 to 3. Length: About 5.2 cm. Aspect: Curved downward. Color: Very light green, 145B to 145C; towards tip, infused with pink.

Peduncles.—Length: About 4.5 to 5 cm. Strength: Strong, flexible. Aspect: Mostly upright. Color: Light green, 143C.

Reproductive organs.—Androecium: Stamen number: Five fused at anthers, hooded; filaments free. Anther shape: Obovate. Anther color: Very light yellow, 19D, with small purple, 57B to 57C, spots. Pollen color: Light pale yellow, close to 8D. Gynoecium: Pistil quantity: Five. Stigma color: Purple, 57C to 57D. Ovary: Five-celled; 5 mm in length. Ovary color: Dark purple, 187A.

Seed development.—Seed development has not been observed.

Disease Resistance: Plants of the new Impatiens have been observed to be resistant to Powdery Mildew.

It is claimed:

1. A new and distinct cultivar of New Guinea Impatiens plant named ‘Kimbu’, as illustrated and described.
